(19) United States
(12) Patent Application Publication
(10) Pub. No.: US 2016/0175612 A1
(43) Pub. Date: Jun. 23, 2016

(54) NAIL FUNGUS LASER TREATMENT

(71) Applicant: FOTONA D.D., Ljubljana (SI)

(72) Inventors: Marko Kazic, Dob (SI); Matjaz Lukac, Ljubljana (SI)

(73) Assignee: FOTONA D.D., Ljubljana (SI)

(21) Appl. No.: 14/578,969

(22) Filed: Dec. 22, 2014

(51) Int. Cl. A61N 5/06; A61B 18/20

(52) U.S. Cl. CPC A61N 5/0624; A61B 18/203; A61N 2005/067

(57) ABSTRACT

A method for treating a fungal infection in an infected nail is disclosed. An embodiment includes placing an optical delivery system designed to deliver a laser beam with light having strongly water-absorbed wavelengths to the infected nail. The diseased nail is irradiated with the laser beam, and the fluence and a duration of the laser irradiation received by the nail are adjusted such that by laser energy absorption in a surface portion of the nail a superficial heating of the nail and heat diffusion from said heated surface portion into the infected nail bed are achieved. The bottom of the nail plate in this embodiment is heated to a treatment temperature needed to inactivate the infecting organism.

### FIELD OF THE INVENTION

The present invention relates to systems and methods for light therapy for use in treatment of nail infections.

### BACKGROUND OF THE INVENTION

Onychomycosis has become a collective term for fungal nail infections, which are most commonly caused by dermatophyte fungi from the genus Trichophyton. It is a public health problem that affects approximately 10% of general population in the US (Westerberg & Voyack, 2013) (estimates from various studies worldwide range from 2-13%) (Elewski, 1998). The risk of infection increases with age: more than 20% of those who are between 60 and 70, and more than 50% of those older than 70 suffer from the condition. The disease also affects a larger proportion (15-40%) of HIV patients. (Westerberg & Voyack, 2013).

Onychomycosis is not merely a cosmetic problem, as it negatively affects patients' emotional, social, and occupational functioning e.g. embarrassment in social situations, employers' reluctance to employ onychomycosis patients in jobs that require food handling or contact with the public. In immuno-compromised people (e.g. HIV positive, chemotherapy patients . . . ) it can lead to serious systemic infections.

Present treatments for onychomycosis include administering systemic and/or local antifungal therapy, surgical nail removal and electromagnetic radiation therapy. Topical administration of antifungal drugs was proven to be largely inefficient in clearing the infection due to their limited ability to penetrate the nail plate. Most commonly used topical drugs are amorolfine and ciclopirox olamine nail lacquer solutions. Their use is recommended as an adjuvant in systemic oral therapy and also as a prophylactic therapy in patients that have previously received a cycle of oral antifungal therapy. New topical drugs with improved nail plate penetration have shown promising results in initial studies (Alley, Baker, Beutner, & Plattner, 2007).

Systemic oral antifungal therapy has been more effective in treating onychomycosis. Commonly used antifungals, itraconazole and terbinafine have shown mycological cure rates from 50% to 80%. However, recurrence rate in monitoring studies up to 3 years of treatment remained high with both drugs—3%-20% for terbinafine and 21%-27% for itraconazole (Tosti, Piraccini, Stinchi, & Colombo, 1998). Because of their systemic and long-term administration, care should be taken to avoid toxic side effects, which in most serious cases can include liver toxicity and heart failure.

In recent years there has been an increase in device based onychomycosis therapy (Gupta & Simpson, 2012). Many of these therapies use electromagnetic radiation aimed at destroying the infective fungus. Prior art electromagnetic radiation devices utilize wavelengths which are at least partially transmitted through the nail and then absorbed in the infected nail bed.

Irradiating the nail with UV light is one among such approaches, which has been disclosed in US20060241729 A1 and U.S. Pat. No. 6,960,201. While UV irradiation works well in eliminating the infectious microorganisms, it can damage the underlying tissue and surrounding skin and is also a known mutagen.

Lasers have also been used as a therapeutic light source for treating nail infections. The existing laser therapies are based on utilizing a wavelength which penetrates substantially through the nail plate and is absorbed in the underlying fungus infected tissue. Absorption of laser energy is then expected to result in sustained heating of the mycelium and fungicidal effects, as fungi can be heat inactivated at temperatures above 40-60° C. Examples of such prior art therapeutic laser wavelengths are a 1064 nm wavelength emitted by an Nd:YAG or a diode laser source, or a 980 nm wavelength emitted by a diode laser source. (Ortiz, Avram, & Wanner, 2014). Typically, laser wavelengths which are not strongly absorbed by the nail are also not strongly absorbed in the infecting fungi. For this reason, the prior art therapeutic laser wavelengths, such as the 1064 nm wavelength penetrate through the mycelium and are absorbed in the underlying tissues, resulting with non-specific bulk heating of the finger. This causes pain and thermal damage to deeper lying healthy tissue.

### SUMMARY OF THE INVENTION

According to the invention, a method for treatment of onychomycosis is proposed, wherein the nail is irradiated by a strongly absorbed laser light wavelength, which is absorbed at the nail surface, and does not get substantially transmitted to the nail bed. This absorption leads to the release of heat at the nail surface, which is diffused through the nail to the underlying nail bed, causing a temperature rise within the nail bed. Laser energy is delivered in the amount necessary to heat the whole thickness of the nail and the upper surface of the nail bed to temperatures that induce inactivation and death of the infecting organism. At the same time, side effects due to tissue damage are minimized, as the heating is directed only at the infected part of the nail bed and does not reach significant levels in healthy underlying tissues. This significantly decreases discomfort and pain compared to standard treatments.

According to the present invention, the rise in temperature is achieved by irradiating the nail with laser light, which needs to be adjusted so that the fluence package that is delivered to the nail is sufficient to achieve a temperature rise throughout the nail plate down to the infected nail bed, while at the same time the laser fluence (energy per area) does not cause significant ablation of the nail surface, but rather mostly thermal effects are achieved on the tissue.

The fluence package can be delivered to the nail either in the form of single pulses or in sequences of multiple pulses. Diode or gas laser most often deliver the laser light in form of a low power long continuous pulses, while solid state (crystal) lasers most often deliver the light in a form of high power short pulses.

When such laser system is used so that the laser light is delivered in the form of high power short pulses (e.g. an Er:YAG laser), the energy that is delivered with fluences that do not induce significant ablation, but only thermal effects, can be too small to achieve the desired temperature rise throughout the nail plate down to the nail bed, which is needed to inactivate the infecting organism.

According to the invention, when such pulsed lasers are used, the pulses can be delivered in single or multiple pulse sequences of multiple non-ablative or low ablative pulses. The parameters of the sequence of pulses are optimally adjusted so that the heat in the underlying nail bed quickly builds up to the temperatures required to kill or inactivate the infecting organism. This, so called, “heat pumping effect” allows temperature build-up below the nail plate without a direct contact or light penetration from the laser source.

In one of the preferred embodiments, the Er:YAG laser may be used. Because the Er:YAG light is absorbed at the nail surface and the heat is diffused through the nail plate into the nail bed, the temperatures reached at the nail bed are longer persisting, and are reached with less bulk heating of the finger when compared with weakly water-absorbing laser wavelengths such as of the Nd:YAG laser source, which is standardly used for laser treatment of onychomycosis. This leads to decreased discomfort during the procedure and higher treatment efficacy compared with the standard Nd:YAG treatment. The treatment is also more energy efficient since only the nail needs to be heated up. In addition, since the temperature increase is localized to the nail and its vicinity, higher treatment temperatures can be used as the remaining underlying tissues remain relatively unaffected.

### DETAILED DESCRIPTION OF THE INVENTION

According to the invention, a method and device for treatment of onychomycosis are proposed, wherein the nail is irradiated by strongly-water absorbed laser light wavelength, which is absorbed at the nail surface. This absorption leads to the release of heat, which is diffused through the nail to the underlying nail bed, causing a temperature rise. Laser fluence (energy per irradiated area by the laser beam) and duration of the laser irradiation are adjusted and delivered in the amount necessary to heat the whole thickness of the nail and the upper surface of the nail bed to temperatures that induce inactivation and death of the infecting organism. A further desired result of said fluence and irradiation duration setting is, that undesired ablation of the nail material is avoided. In other words no, no significant or only minimal nail material ablation takes place. The entire nail is kept in its previous general shape and general thickness.

The fluence package can be delivered to the nail either in the form of single pulses or in sequences of multiple pulses. Diode or gas laser most often deliver the laser light in form of a low power gated continuous output, while solid state lasers most often deliver the light in a form of high power short pulses.

When such laser system is used so that the laser light is delivered in the form of high power short pulses (e.g. an Er:YAG laser), the energy that is delivered with fluences that do not induce significant ablation, but only thermal effects, can be too small to achieve the desired temperature rise throughout the nail plate down to the nail bed, which is needed to inactivate the infecting organism.

According to the invention, the presented system and method are used for a laser-based thermal treatment of the nail and the infected nail bed, which does not cause significant ablation of the nail surface. Our invention is based on the concept of controlled heat deposition or introduction into the nail.

According to the invention, the inactivation of the infecting organism is due to heat-related damage that occurs after the infecting fungi were exposed to temperatures higher than 40-60° C. The heat is delivered to the infected nail bed by heat diffusion from the nail plate, which is irradiated by a strongly laser light. The fluence package, or cumulative energy per area irradiated by the laser beam, needs to be large enough to allow the temperature rise in the whole nail plate, so that the infected nail bed below is heated as well, and a “hot iron” effect is reached.

The thickness of healthy nails (s) varies, with the thickness typically within the range of 0.2 mm to 1 mm Infected nails may be thicker, up to 3 mm, or more. This means that heat deposition has to diffuse through maximally 3 mm thick or even thicker nails down to the infected nail bed, but further heating of the underlying tissues need to be minimized In order to achieve the controlled heat deposition into the nail bed, an effective and safe heat source is needed, which is capable of distributing heat through the nail to the infected tissue located just beneath the nail, without damaging neither the nail nor the deeper lying surrounding tissues.

The energy needed to achieve the desired temperature rise throughout the nail plate depends on the heat needed to achieve the temperature change at the nail base because the laser energy is completely absorbed in the surface layers of the nail plate and is released as heat.

Our experiments using an Er:YAG laser showed that the absorbed energy per irradiated nail plate area that is needed to increase the temperature of the nail base by 40° C. is in a range of approximately 25 J/cm² for a 1 mm thick nail to approximately 110 J/cm² for a 3 mm thick nail.

Because the nail plate contains 7-25% of water, lasers with strongly water-absorbed wavelengths, in a wavelength range from above 1.9 to 11 microns, such as Tm:YAG (wavelength of 2.0 microns), Ho:YAG (wavelength of 2.1 microns), Er:YAG (2.9 μm), other erbium ion doped laser types including Er,Cr:YSGG, Er:YSGG, Er:YAP or Er:YLF (2.7 to 3.0 μm) or CO2 (from about 9.3 to about 10.6 μm) laser source, are especially useful to achieve this non-contact “hot iron” heat source effect, where the laser light is absorbed on the nail surface and the energy is released as heat deeper into the nail. Absorption depths of these laser sources in the nail are in the range from 1 to 100 microns (Er:YAG: 1 microns; Er,Cr:YSGG: 3 microns; CO2: 10 microns and Ho:YAG or Tm:YAG: 100 microns), which ensures that the laser light energy is absorbed effectively and safely within the nail plate.

In order to understand our invention, one must realize that there are four steps in heating when the nail is exposed to water-absorbed laser radiation. The nail is first heated directly (first step) within the optical absorption depth dₒₚₜ of approximately 1-100 μm (Er:YAG : about 1 μm, Er,Cr:YSGG: about 3 μm, CO2: about 10 μm, Tm:YAG or Ho:YAG: about 100 μm).

Direct heating is followed by thermal diffusion (second step) during the pulse that indirectly heats the deeper lying nail layers. For short pulses, the time span for thermal diffusion during a pulse is short; the heat energy therefore does not reach very deep into the nail. For longer pulses, the heat has sufficient time during the pulse to propagate deeper into the nail.

The third step occurs only when the laser pulse fluence is sufficiently high to heat the thin surface layer up to the water evaporation or to the bulk nail melting temperature. This would lead to the ablation of the superficial nail layers. It is to be noted that lasers with strongly water-absorbed wavelengths are known in the art as ablative lasers, due to their very effective and fast ablation of organic tissues. But it is our goal to achieve heating of the nail tissue while minimizing ablation by means of our inventive method and system.

The fourth step occurs following the end of the laser pulse when thermal diffusion continues to indirectly heat the deeper laying nail layers.

As only (or mostly) thermal effects are desired, the fluence of the laser beam (which is usually calculated in J/cm²) must not be significantly higher than the ablation threshold. The ablation threshold depends on the laser wavelength and pulse duration, and is lower for more strongly absorbed laser wavelengths and for longer pulse durations.

For short pulse durations below approximately 100 microseconds where the effect of heat diffusion during the pulse is negligible, the minimal ablation threshold fluence (F₁) can be estimated from F₁=hₐ×dₒₚₜ, where hₐ is the specific heat of ablation (in J/cm³). For the human nail, the specific heat of ablation is approximately hₐ≈1 J/mm³, resulting in approximate minimal ablation threshold fluences (F₁) of 1 J/cm² for Er:YAG, 3 J/cm² for Er,Cr:YSGG, 10 J/cm² for CO2, and 100 J/cm² for Tm:YAG.

At longer pulse durations, the ablation threshold is expected to increase because of the conductive loss of heat from the absorption depth layer during the second step of the heating cycle. However, in terms of laser efficiency, it is preferable to operate flashlamp or diode pumped lasers in short laser pulses, up to maximally several milliseconds, and preferably up to 2 milliseconds.

The characteristic diffusion depth (x_{D}) to which the tissue temperature is affected after a time interval t can be estimated from x_{D}=√(Dt), where D is the nail thermal diffusivity of about D=2 .10-7 m2/s. During the pulse duration of 2 milliseconds, for example, the diffusion depth is about x_{D}=20 μm. The ablation threshold fluence (F₂) for a case when x_{D}>dₒₚₜ can be then estimated from F₂=F₁ √(x_{D}/dₒₚₜ). For a 2 milliseconds long pulse, the approximate ablation threshold fluences (F₂) are 4 J/cm² for Er:YAG, 8 J/cm² for Er,Cr:YSGG, and 14 J/cm² for CO2, while the ablation threshold for Tm:YAG or Ho:YAG remains unaffected by diffusion (F₂≈F₁=100 J/cm²). Our experiments with 0.6 milliseconds long Er:YAG laser pulses show that the ablation threshold fluence varies from nail to nail, and is typically within the range of 1 to 5 J/cm².

It follows from the above that under most circumstances it is not possible to heat up the nail base up to required treatment temperatures, while minimizing the ablation of the nail surface, using a single short pulse laser. Since the above ablation threshold fluences are much lower than the required energy per irradiated area, either very long pulses of sub- or minimally ablative fluences should be used, or more consecutive pulses should be shot on the same area, with the pulses optimally spaced apart to achieve cumulative heat disposition (“heat pumping”) on the irradiated area.

Appropriate laser parameters depend on the type of laser system used and the specific treatment indication.

The abovementioned principle is employed in the preferred embodiment of the present invention, when solid state Er:YAG laser source is used, which is preferably flash lamp or diode pumped and therefore operated in a pulsed mode. In terms of laser efficiency, it is preferable to operate the Er:YAG laser in short laser pulses, up to millisecond range. The energy per irradiated area delivered by such single Er:YAG pulse is too low to increase the temperature of the nail plate to the treatment level, when minimal ablation is desired. Increasing the energy delivered in a short pulse would lead to higher fluences and unwanted ablation of tissue.

According to the invention, when higher doses of heat are required to be delivered to the nail by a laser operating in pulsed mode, in order to heat up the infected tissues just beneath the nail plate, a special “smooth” pulse sequence delivery mode is proposed. In said “smooth” pulse sequence mode the energy is delivered to the nail in a consecutive sequence of several individual laser pulses wherein the fluence of each of the individual laser pulses in the sequence is below or close to the ablation threshold. When the temporal separation among the individual pulses, that is the pulse separation time tₚₛ, is longer than the thermal relaxation time TRT_{surface} of the nail surface tissue (estimated to be in the range of 10-50 ms), the nail surface has sufficient time to cool between the pulses by dissipating the heat deeper into the nail during the fourth step of the heating cycle. The TRT is the time required for the tissue temperature to decrease by approximately 63%. Thus, in the case when the temporal separation time tₚₛ, is longer than the TRT_{surface} of the nail surface, the temperatures required for ablation of the surface of the nail are reached at much higher cumulative fluences compared to the fluence of an individual pulse, because the diffusion of heat deeper into the nail plate, which prevents the nail surface temperature from getting dangerously elevated. And if at the same time laser energy is delivered in a time period that is shorter than the TRTₙₐᵢₗ of the total nail plate (estimated to be in the range of 5-10 s) then the deeper lying nail layer does not have time to cool off during the laser pulse sequence. The delivered laser energy thus results in an overall non-ablative or minimally ablative build-up of heat and creates a temperature increase throughout the thickness of the nail and at the top layer of the infected nail bed without significantly damaging the nail surface.

According to the invention, the laser energy can be delivered to the nail either continuously, or, preferably, in the form of single laser pulses or sequences of multiple pulses.

For strongly water-absorbed laser wavelengths the single pulse fluence range needed to achieve the desired non-ablative or minimally ablative heating shall vary from nail to nail, and will be depending also on the laser wavelength within the range of 0.2 to 150 J/cm².

The above fluence range as claimed allows the laser to operate as high as possible above the lasing threshold since the laser becomes very inefficient (in terms of laser energy output vs. input) when it operates close to the lasing threshold. With fluences outside the claimed range, the method according to the invention would become impractical and/or extremely difficult to realize. The upper limit of the above fluence range is chosen to ensure that the temperature of a 3 mm thick nail can be increased for about 40° C. by a single pulse, and at the same time that the single pulse fluence is close to or below the ablation threshold for laser wavelengths with optical absorption depth at or above 100 nm.

It may be more energy efficient for a particular laser configuration to operate in a pulse sequence consisting of a relatively small number (N) of pulses separated by a relatively short pulse separation time (tₚₛ). In such a case the duration of the irradiation of the target area on the nail during which the required cumulative fluence is delivered may be prolonged by delivering laser energy in multiple pulse sequences. In a preferred embodiment, multiple pulse sequences may follow each other with a sequence separation time T_{Ss} in a range from 0.2 s, inclusive, to 2.0 s, inclusive, and preferably at least approximately of 0.5 s.

We have determined that with our method the bottom surface of the nail that is facing the infected nail bed can be heated to temperatures in the range from 40 to 80 degrees Celsius, which is the temperature that is detrimental to infecting fungi. By controlling the thermal diffusion depth by using different pulsing schemes, the treatment focuses on the infected area just below the nail plate. It also allows for the system to be highly tunable to differences in the thickness of the nail plate (e.g. toenails are generally thicker than finger nails; there are also great differences between individuals).

According to the invention a laser system is proposed, comprising a laser source for generating a laser beam and a control unit and a hand piece for manually, or using a scanning device, guiding the laser beam onto the nail surface, wherein a wavelength of the laser beam is strongly water-absorbed, in a range from above 1.9 μm to 11.0 μm inclusive, and wherein the laser system including the control unit is adapted for a thermal, non-ablative, or minimally ablative treatment of infected nails by means of the laser beam such, that the laser source generates the laser beam in single pulses with a pulse duration in a range from 1.0 μs (microseconds), inclusive, to 10 s (seconds), inclusive, and that a fluence of a single individual pulse on a target area of the nail plate is in a range from 0.2 J/cm², inclusive, to 150 J/cm², inclusive, and preferably in a range from 0.5 J/cm², inclusive, to 10 J/cm², inclusive, wherein the fluence and the duration of the laser beam are adjusted so that the energy delivered to the nail is sufficient to heat the entire nail plate to the desired temperature.

The above maximal 10 s single pulse duration is chosen in order for the pulse to be not considerably longer than the TRTₙₐᵢₗ of the total nail (5-10 s), and that the diffusion depth during this pulse duration is not much longer than the typical nail thickness of about 1 mm.

In order to prove and to provide evidence that our invention achieves the desired effect, measurements have been carried out, showing the achieved temperature of the nail is dependent on the fluence on the target area and on the number of single pulses within one pulse sequence. The measurements were carried out with a thermal camera having a temporal resolution of 20 msec. During this time, the superficially absorbed laser energy thermally diffuses approximately 60 μm deep into the tissue. In addition, since the camera software assumes a uniform body temperature, the measured temperatures represent a weighted average of the nail temperature within the penetration depth of the detected thermal radiation of approximately 100 μm. Thus, even though experiments were made with an Er:YAG laser, the results apply also to other highly absorbed laser wavelengths, with optical absorption depths dₒₚₜ at or below about 100 μm.

We have carried out different measurements in order to compare and provide evidence that the present invention discloses superior system and method for the treatment of onychomycosis than commonly used Nd:YAG method. In the first set of experiments, we wanted to examine in vitro whether the Er:YAG system and method can efficiently raise the temperature on the bottom side of a human nail. Nail clippings of 0.45 mm thickness were exposed to pulses from an Nd:YAG laser source with the following standard onychomycosis treatment parameters—pulse duration 30 ms, 6 mm beam diameter, 25 J/cm² single pulse fluence. When irradiated with a single pulse of the Nd:YAG laser light with above parameters, the back surface temperature of the nail increased by about 40° C. We wanted to reach similar temperature changes by using the novel system and method using an Er:YAG laser source. The Er:YAG laser beam was delivered in several subsequent or consecutive “smooth” pulses, each smooth pulse consisting of a sequence of five tₚ=0.6 msec long pulses of equal energy within the overall smooth mode pulse duration of 203 msec, the temporal separation between the pulses (tₚₛ) thus being equal to 50 msec. The Er:YAG laser beam spot size was 6 mm.

The cumulative fluence of the smooth mode pulse sequence was varied from 2 to 4 J/cm², the number of successively delivered smooth mode pulse sequences was varied from M=1 to 4, and the repetition rate (1/T_{Sr}) of the smooth mode pulses was varied from 0.5 to 1.5 Hz. The nail clipping was irradiated at the front surface, and the temperature of the nail's back surface that was not directly irradiated was monitored immediately following the irradiation. When the cumulative fluence of the smooth mode pulse was set to 3 J/cm² (resulting in the fluence of each individual pulse within the smooth pulse sequence of 0.6 J/cm²), and there were three smooth mode pulses delivered to the nail with a repetition rate of 1 Hz, the back surface temperature of an s =0.51 mm thick nail increased by about 39° C. Thus, the 3×3 J/cm²=9 J/cm² of cumulatively delivered Er:YAG laser fluence resulted in approximately the same temperature increase on the back side of the nail as obtained with approximately three times larger (25 J/cm²) laser fluence of the Nd:YAG laser.

To determine the effect of nail thickness on the temperature increase after laser treatment, nail clippings of various thickness were exposed to Er:YAG light (single 203 msec long smooth mode pulse consisting of five individual pulses, smooth mode fluence 3 J/cm², beam diameter 6 mm) The results show that when the same laser parameters are used, the temperature increase on the back side of the nail is much greater in thinner than in thicker nails. In a series of experiments we have determined the following approximate cumulative fluences of strongly water absorbed laser wavelengths that are required to increase the temperature on the back side of the nail by 40° C.: 3 J/cm² for s=0.3 mm; 9 J/cm² for s=0.5 mm; 25 J/cm² for s=1 mm; 67 J/cm² for s=2 mm; 110 J/cm² for s=3 mm.

To summarize, the comparison has shown that using the Er:YAG system and the disclosed pulsing scheme, the same temperatures on the nail surface facing the nail bed can be reached while using much lower fluences. In addition, the heat on the back surface of the nail plate persisted much longer when the Er:YAG system and method were used. Temperatures on the back nail surface persisted above 50° C. for 5s after each Er:YAG pulse sequence, which was about 20 times longer than the average temperature increase duration following a standard Nd:YAG treatment pulse. Also, by shortening the time between the pulse sequences, so the tissue is not allowed to cool down before the next pulse, a so-called “heat pumping” effect can be enhanced—maximum nail temperature can be further raised with each subsequent pulse.

In the second set of experiments aimed to determine in vivo whether using the Er:YAG treatment system and method provides any advantages for the patient in terms of decreased pain sensation. Nails of a human volunteer were treated using a standard Nd:YAG method and the novel Er:YAG method. Nail surface temperature was monitored during the experiments. The results have shown that the treatment with the Er:YAG laser allows a much larger increase of the top nail surface temperatures , up to 95° C., before any sensation of pain. This temperature was reached, for example, when the nail was exposed to an overall cumulative fluence of 24 J/cm² during the smooth mode sequence duration of 8 seconds. In contrast, the Nd:YAG laser treatment resulted in pain sensation when the top nail surface temperature was increased only to 50° C. (which was reached following 4 subsequent, 35 J/cm2 Nd:YAG laser pulses delivered with 1 Hz repetition rate). These differences are attributed to the difference in the depth of laser penetration. The Er:YAG 2940 nm laser wavelength is absorbed at the nail plate surface where heat is released and diffused to the nail bed below while leaving the underlying tissues unheated. In contrast, the Nd:YAG laser penetrates through the nail and deeper into the tissue, thus heating up also the non-infected underlying tissues, the effect which is not therapeutically indicated. Because the infected tissue lies directly below the nail plate, the Er:YAG method is a precise and most direct way to reach the infected bottom surface of the nail bed without damaging the healthy tissues below.

The in vivo measured TRT of the Er:YAG laser irradiated nail surface was in the range of TRT_{surface}=10−50 msec, while the measured TRTₙₐᵢₗ of the complete nail plate was depending on the thickness of the irradiated nail in the range of TRTₙₐᵢₗ=5−10 seconds.

In summary, the disclosed system and method present a non-contact “hot iron” source, bringing together the advantages of direct heating of the nail plate (which minimizes heating of the deeper lying tissue) with the advantages of using a non-contact laser source. The non-contact laser heating source does not require a good thermal contact of the nail with the heating source. In addition, when using collimated laser beams, the laser heat pumping is relatively insensitive to the spatial separation of the laser source output optics with regard to the treated nail. Using the inventive laser “hot-iron” source also allows the precision in determining the duration of heat pumping, by controlling the duration and temporal structure of the laser irradiation. Moreover, the inventive heat pumping enables easy adjustment and control of the treatment area by varying the beam spot size, and therefore the irradiation area, along with various options for monitoring and control.

In one embodiment, the inventive device and method is used in such a way that by increasing the laser fluence above the ablation threshold the same laser device is first used for the pre-treatment “ablative” step to ablate down the nail to a smaller thickness, followed by the non-ablative heat pumping treatment step using sub-ablative laser fluences. In yet another embodiment, both steps: the laser pre-treatment ablative step, and the treatment heat pumping step may be joined into a single joint step by setting the laser fluence to a value at which the ablation of the nail and the thermal heat diffusion through the nail plate can be achieved to a sufficient degree simultaneously.

In certain embodiments, a laser-scanning device is added, which scans the laser beam across the nail. The scanning device may be capable of detecting the surface of the nail plate.

Additionally, an IR temperature sensor may be included to measure the temperature of the nail surface, and then used as a feedback to achieve uniform and/or optimal heating of the nail plate. The laser beam would be kept fixed at the same irradiation area and laser irradiation applied until the surface nail temperature reached 70-80° C., which according to our experiments are not painful for the patient.

Further, a combined laser wavelength treatment may be performed using two laser sources, one with a water-absorbed wavelength, and the other with a transmitted, non-water-absorbed wavelength which is at least partially transmitted through the nail, whereas the nail bed is first pre-heated with a strongly water-absorbed wavelength to temperatures from 40 to 80° C. according to the inventive method described above, followed by an irradiation with the not strongly water-absorbed laser wavelength. In a preferred embodiment, the wavelength of the not strongly water-absorbed laser wavelength is in a range from 0.35 μm to below 1.9 μm with the pulse duration in a range from 0.5 ns to 50 ms, and the laser fluence (of one pulse) is in a range from 1 J/cm² to 150 J/cm². The temporal separation between the two types of irradiations should be shorter than the measured thermal relaxation time of the complete nail plate (TRTₙₐᵢₗ) in the range from 5 s to 10 s, preferably shorter than 1.0 s, inclusive. The not strongly water-absorbed wavelength irradiation may be delivered also at least partially during the time when the strongly water absorbed wavelength irradiation is being delivered. A picosecond duration (1-999 μs) laser, a Q-switched nanosecond pulse duration (1-200 ns) laser, a microsecond laser (1-999 p), or a millisecond duration (1-300 ms) laser with a transmitted laser wavelength may be used. In one of the preferred embodiments the not strongly water-absorbed wavelength is that of the Nd:YAG laser (1064 μm), or Nd:YAP laser (1079 μm). In another embodiment, a diode laser may be used with a wavelength in the range of 800 nm to 1100 nm. The advantage of this combined wavelength device and method is that the temperature of the fungus infected tissue is first raised up to detrimental temperatures for the fungi without significantly affecting the temperature of underlying tissues, and thus creating a “temperature shock” to the fungi, and then submitting the fungi to the second, “optical shock” by irradiating the fungi directly with the second transmitted laser wavelength, thus utilizing the additional germicidal effect of the direct illumination by the electromagnetic radiation. Since the fungi has been pre-heated, the optical shock treatment can be performed with lower parameters of the transmitted laser light as compared to those that would have to be used with a standard (prior art) single transmitted wavelength method. Alternatively, due to the pre-heating, the treatment efficacy with the prior art transmitted wavelength parameters is enhanced.

It is to be appreciated that the above inventive device and method can be used also for treating other microbial skin infections and not just for treating nail infections.

### BRIEF DESCRIPTION OF THE DRAWINGS

Exemplary embodiments of the invention will be explained in the following with the aid of the drawing in more detail. With reference to the following description, appended claims, and accompanying drawings:

FIG. 1 illustrates an exemplary inventive laser device with both an optical fiber laser delivery system and an articulated arm laser delivery system;

FIG. 2 illustrates in a schematic view a fungus infected nail under irradiation by means of the laser device according to FIG. 1; and FIG. 3 illustrates an exemplary pulse sequence of the laser beam generated by the laser device according to FIG. 1, and delivered to the infected nail according to FIG. 2.

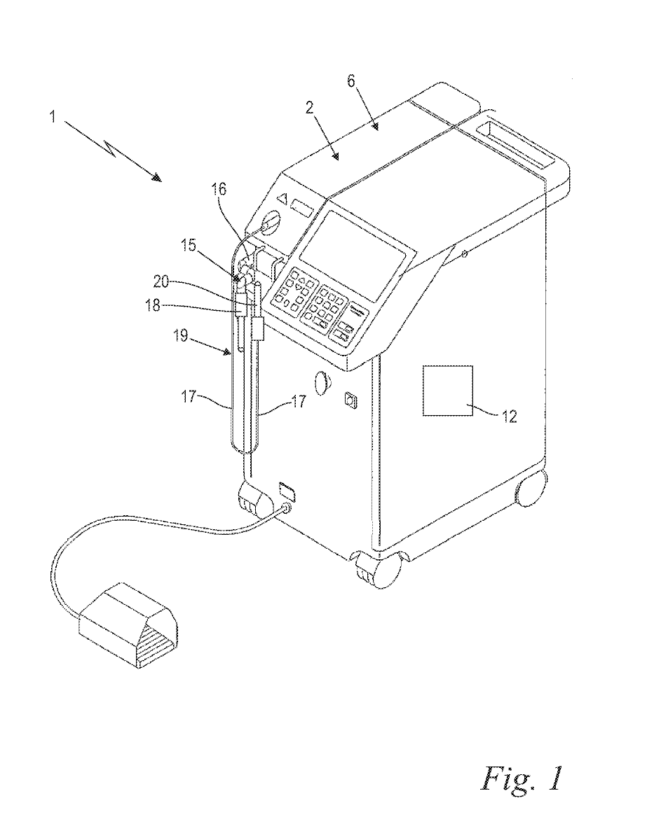

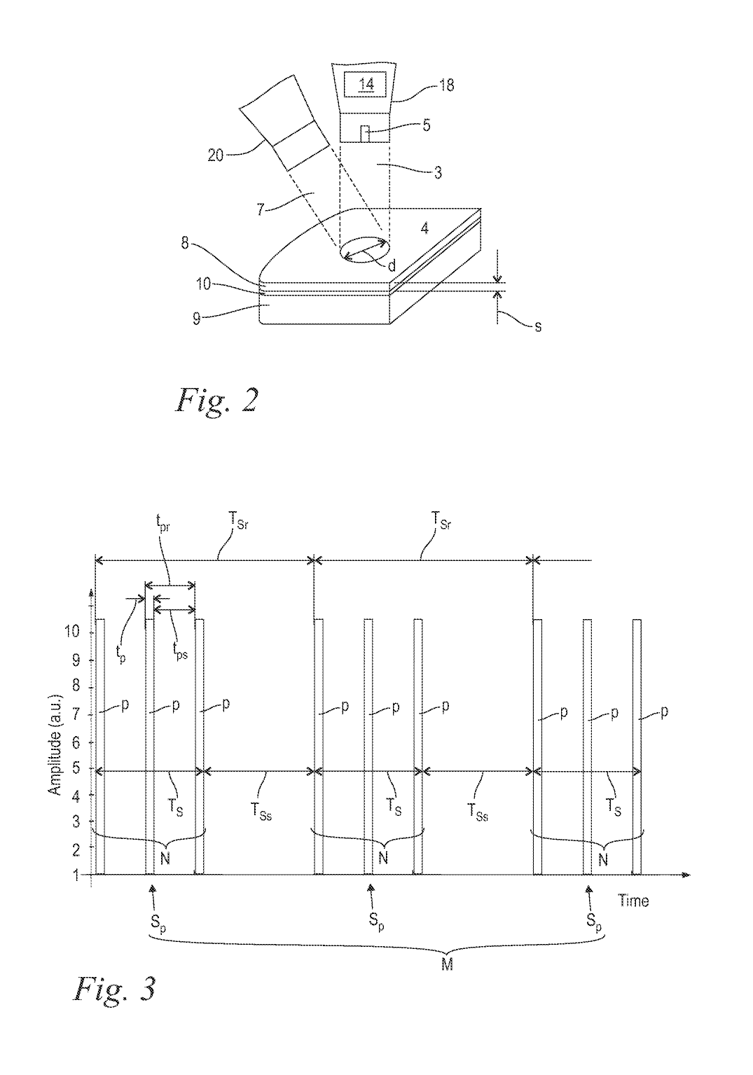

With reference now to FIG. 1 a medical treatment laser device 1 according to the invention comprises at least one laser system 2, 6 for generating a laser beam 3, 7 (FIGS. 2, 3), and at least one optical delivery system 15, 19 for the laser beam 3, 7. In the shown preferred embodiment the laser device 1 comprises two integrated individual laser systems 3, 6, each having an individual laser source. The laser device 1 further comprises a schematically indicated control unit 12 for controlling the operation of the at least one laser source of the at least one laser system 2, 6, including the generated laser beam 3, 7 parameters. In the shown embodiment, the control unit 12 controls the operation of both laser sources and is therefore integral part of both laser systems 2, 6. However, each laser system 2, 6 may have an own individual control unit 12.

In one preferred embodiment the first optical delivery system 15 includes an articulated arm 16 and a manually guided laser treatment head 18 connected to the distal end of the articulated arm 16, wherein the laser beam 3, 7 is transmitted, relayed, delivered, and/or guided from either one or both laser systems 2, 6 through the articulated arm 16 and the laser treatment head 18 to a target. The articulated arm 16 might preferably be an

Optoflex® brand articulated arm available from Fotona, d.d. (Slovenia, EU). In the shown preferred embodiment a second optical delivery system 19 is provided, wherein instead of the articulated arm 16 a flexible elongated delivery fiber 17 for guiding the laser beam 3, 7 from either one or both laser systems 2, 6 is incorporated. As part of the second optical delivery system 19 a manually guided laser treatment head 20 is attached to the distal end of the elongated fiber 17. Despite both laser systems 2, 6 and delivery systems 15, 19 being shown in combination, at least the first laser system 2 with a related delivery system 15, 19 may be provided and used within the scope of the present invention. Either one of the optical delivery systems 15, 19 might be used in connection with either one of the laser systems 2, 6, thereby guiding either one or both of the laser beams 3, 7.

Alternatively, one or both laser sources may be built into the laser treatment head 18, 20, whereas the laser treatment head 18 itself represents one or both optical delivery systems 15, 19 for the laser beam 3, 7. Moreover, the control unit 12, or a complete medical laser system 2, 6 may be built into the laser treatment head 18, 20 as well.

According to the invention, the wavelength of the laser beam 3 generated by the first laser system 2 is in a range from 1.9 μm, exclusive, to 11 μm. It is known, that within said wavelength range, such laser beam is highly absorbed in water, when passing water or a water containing medium. The present laser beam 3 of the laser system 2 is here therefore referred to as a strongly water-absorbed laser beam. In the embodiment shown in FIG. 1 the strongly water-absorbed laser beam 3 of the first laser system 2 is generated by an Er:YAG laser system and exhibits a wavelength of 2.94 μm. However, the laser system 2 can also be a Tm:YAG laser system generating a first, water-absorbed laser beam having a wavelength of 2.0 μm, a Ho:YAG laser system generating a strongly water-absorbed laser beam having a wavelength of 2.1 μm, a Er,Cr:YSGG or a Er:YSGG laser system generating a strongly water-absorbed laser beam having a wavelength in a range from 2.73 μm to 2.78 μm, an erbium ion doped laser system, preferably an Er:YAP or an Er:YLF laser system, having a wavelength in a range from 2.7 μm to 3.0 μm, or a CO2 laser system generating a strongly water-absorbed laser beam having a wavelength in a range from 9.3 μm to 10.6 μm.

According to the invention, the wavelength of the laser beam 7 generated by the second laser system 6 is in a range from 0.35 μm, inclusive, to 1.9 μm, inclusive. It is known, that within said wavelength range, such laser beam is poorly absorbed in water, when passing water or a water containing medium. The present laser beam 7 of the second laser system 6 is here therefore referred to as a not strongly water-absorbed laser beam.

In the embodiment shown in FIG. 1 the not strongly water-absorbed laser beam 7 of the second laser system 6 is generated by a Nd:YAG laser and exhibits a wavelength of 1064 nm. However, the second laser system 6 can also be a Nd:YAP laser system generating a second, not strongly water-absorbed laser beam having a wavelength of 1079 nm. The second laser system 6 can be picked from one of the following laser types: a picosecond duration laser, a Q-switched nanosecond pulse duration laser, a microsecond laser or a millisecond laser.

FIG. 2 shows in a schematic perspective view a fungus infected human finger or toe nail 8 during application of the inventive laser treatment method. The nail 8 having a thickness s is supported by a nail bed 9. In a fungus infected state, a fungus layer 10 is located below the nail 8, in other words between the nail 8 and the nail bed 9. For treating said infected nail 8, and as further shown in FIG. 2, the strongly water-absorbed laser beam 3 of the laser device 1 is applied to the infected nail 8 by directing the related and schematically shown treatment head 18 onto the nail surface. The laser beam 3 is focused to generate an irradiation spot 4 with a mean diameter d on the nail 8. An irradiation spot 4 having a circular shape is shown as an example. However, any other suitable shape of the irradiation spot 4 might be chosen as well. Shape and size of the irradiation spot 4 may not suffice to irradiate the entire nail 8. For irradiation of the entire nail 8, or at least the infected portion thereof, the treatment head 18 may be manually guided such, that the entire target area is subsequently covered by said radiation spot 4. In the alternative, suitable guiding means like a schematically indicated scanner 14 may be used to achieve the desired irradiation of the entire target area.

By nature, the nail 8 has a certain water content, on which the present invention relies when irradiating the infected nail 8 by the water-absorbed laser beam 3. As the thickness s of the nail 8 is typical between 0.3 mm and 3 mm, and due to the inherently present water content of the nail 8, the major portion of the water-absorbed laser beams 3 energy is absorbed within a penetration depth in an absorption layer close to the outer surface of the nail 8. The penetration depth for the Er:YAG laser beam as used in the illustrated embodiment is typical 1 μm. In any case the absorption depth is only a small fraction of the nail thickness, in consequence of which the fungus layer 10 is virtually not at all subjected to any direct irradiation by the strongly water-absorbed laser beam 3. However, and due to said absorption, the laser energy absorbed within the penetration depth is converted into heat energy, which instantaneously heats up the superficial absorption layer of the nail 8. Said heat energy is furthermore transmitted from the superficial absorption layer of the nail 8 to the deeper lying layers of the nail 8 and finally, after some time delay, to the fungus layer 10 via heat diffusion. As a result, and under particular consideration of laser parameters as described below, both the entire nail 8 and the underlying fungus layer 10 are heated up to certain target or treatment temperature.

For achieving the desired treatment temperature, the first laser system 2 is operated under the control of the control unit 12 (FIG. 1) in a pulsed operation mode, thereby generating the strongly water-absorbed laser beam 3 in individual pulses p (FIG. 3) with a first parameter set of laser parameters. One, some or all parameters of the first set of parameters is/are adjusted such, that the infected nail 8 is irradiated in a non- or low ablating manner and thereby heated to a treatment temperature in a predefined range. In the embodiment according to the FIGS. 1-3 the target or treatment temperature is in the predefined temperature range from 40° C., inclusive, to 80° C., inclusive. In a preferred embodiment the treatment temperature is in a range from 60° C., inclusive, to 80° C., inclusive. The temperature of the nail 8 as achieved by said laser irradiation is optionally monitored by means of a temperature sensing device. In the embodiment according to FIG. 2 the temperature sensing device is an infrared temperature sensor 5.

In the embodiment according to FIG. 2 the first set of parameters is adjusted to heat the nail 8 to the aforementioned temperature ranges in response to the signal of the temperature sensing device. The temperature sensing device may optionally be connected to the control unit 12 (FIG. 1) to form a closed loop control circuit for achieving and keeping a certain and predetermined target or treatment temperature.

The first laser system 2 of the embodiment shown in the FIGS. 1-2 is operated in pulsed operation mode. In FIG. 3 the intensity of laser light generated by the first laser system 2 of the laser device 1 is plotted schematically over the time. This intensity-time plot shows a number of individual pulses p and their temporal relationship to each other for demonstrating basic laser parameters of said first parameter set, as described in the following under reference to FIG. 3. For the sake of simplicity the pulses p are plotted with a rectangular shape. However, in reality each pulse p has an initial rising slope up to a peak value, followed by a subsequent declining tail slope. As one of the first parameter set each individual pulse has a pulse duration tₚ given by the temporal width between the onset of the initial rising slope and the termination of the declining tail slope. Individual pulses p may be grouped in at least one pulse sequence Sₚ. The number of individual pulses p that are part of one pulse sequence Sₚ is called individual pulse number N. In the shown example three subsequent pulses p are forming one pulse sequence Sₚ, thus the individual pulse number N is three. The individual laser pulses p of one pulse sequence Sₚ are temporally separated by a pulse separation time tₚₛ and follow one another in a pulse repetition time tₚᵣ. During the pulse separation time tₚₛ the output intensity of the laser system 1 is negligible or even zero. The time from the beginning of the first individual pulse p of one pulse sequence Sₚ to the end of the last individual pulse p of the same pulse sequence Sₚ is called pulse sequence duration Tₛ.

In a preferred embodiment of the invention, multiple pulse sequences Sₚ subsequently follow one another. Three subsequent pulse sequences Sₚ each comprising three individual pulses p are shown in FIG. 3 as an example. Thus the sequence number M, giving the number of sequences within one uninterrupted application of the laser beam 3 on the nail 8, is three in the example presented in FIG. 3. The pulse sequences Sₚ are temporally separated by a sequence separation time T_{Ss} and follow one another in a sequence repetition time T_{Sr}. Again, during the sequence separation time T_{Ss} the output intensity of the laser system 1 is negligible or even zero. Within one uninterrupted application of the laser beam 3 on the nail 8 a total pulse number K=N×M is delivered, with N=3 and M=3 leading to a total pulse number K=9 in the present example.

In addition to said parameters a further important parameter is the fluence F The afore mentioned parameters of the first laser parameter set for the operation of the first laser system 2 as described along with FIG. 3 are chosen to heat the nail 8 and in consequence the underlying fungus layer 10 (FIG. 2) by multiple pulse “heat pumping” and heat diffusion, while at the same time avoiding ablation of the nail surface. An important physical parameter limiting the selection of said parameters for operation of the first laser system 2 is the fluence F delivered by one individual laser pulse p. The fluence F is the amount of energy delivered per unit area. In other words the fluence F is the amount of energy delivered by one individual pulse p (FIG. 3) and distributed over the area of the irradiation spot 4 on the nail 8 (FIG. 2). To avoid ablation of the nail 8 the fluence F of one individual pulse p is kept small enough to remain under an ablation threshold. The ablation threshold resembles the upper fluence limit for the inventive fungus treating method. However, for energetic efficiency reasons the laser system should be operated as far above the lasing threshold as possible. Therefore the output power should not be too small. As the output power of a laser is correlated with the fluence F, the usable fluence range has within the invention a lower limit.

One aspect to adjust the fluence to the required level is choosing or adjusting an appropriate area which is irradiated during the course of on individual pulse p. One preferred option to achieve this goal is to keep the mean diameter d of the irradiation spot 4 (FIG. 2) in a range from 4 mm, inclusive, to 8 mm, inclusive, and in particular at approximately 6 mm.

According to one aspect of the invention, and in order to achieve the goal of an effective, non- or low ablative nail heating, the nail 8 might be irradiated with one or more single, individual pulses p. Such one or more single, individual pulse p preferably has a pulse duration tₚ in range from 1 μs, inclusive, to 10 s, inclusive. The area of the irradiation spot 4 and laser parameters of the first parameter set are chosen and adjusted such that the fluence F (FIG. 2) generated by one individual laser pulse p (FIG. 3) of the first, water-absorbed laser beam 3 within the irradiation spot 4 on the nail 8 is in a range from 0.2 J/cm², inclusive, to 150 J/cm², inclusive. In a preferred embodiment said fluence F is adjusted to be in a range from 0.5 J/cm², inclusive, to 10 J/cm², inclusive. In another preferred embodiment the fluence F is adjusted to be >0.6 J/cm².

In the embodiment shown in FIGS. 1-3 the pulse duration tₚ of the first laser system 2 operated with a first set of parameters is in a range from 1 μs, inclusive, to 10 s, inclusive. In a preferred embodiment the pulse duration tₚ of the first laser system 2 operated with a first set of parameters is in a range from 10 μs, inclusive, to 2000 μs, inclusive. In another preferred embodiment the pulse duration tₚ of the first laser system 2 operated with a first set of parameters is approximately 600 μs.

According to another aspect of the invention, and in order to achieve the goal of an effective, non- or low ablative nail heating, the nail 8 might be irradiated with one or more pulse Sequence Sₚ. Within the scope of this document a pulse sequence Sₚ meeting the below defined requirements is also referred to as a “smooth pulse”. In the embodiment shown in FIGS. 1-3 the pulse sequence duration Tₛ of such pulse sequence Sₚ generated by the first laser system 2 operated with a first set of parameters is ≦10 s and the pulse separation time tₚₛ is ≧10 ms. In a preferred embodiment the pulse sequence duration Tₛ is in a range from 1 μs, inclusive, to 10 s, inclusive. In another preferred embodiment the pulse sequence duration Tₛ is in a range from 1 μs, inclusive, to 1.5 s, inclusive. In another preferred embodiment the pulse sequence duration Tₛ is 0.25 s.

In the embodiment shown in FIGS. 1-3, and within said “smooth pulse”, the pulse separation time tₚₛ of the first laser system 2 operated with a first set of parameters is preferably in a range from 0.01 s, inclusive, to 2 s, inclusive. In a preferred embodiment the pulse separation time tₚₛ of the first laser system 2 operated with a first set of parameters is in a range from 0.05 s, inclusive, to 0.2 s, inclusive.

In the embodiment shown in FIGS. 1-3, and within said “smooth pulse”, the pulse number N of the first laser system 2 operated with a first set of parameters is in a range from four, inclusive, to eight, inclusive. In a preferred embodiment the pulse number N of the first laser system 2 operated with a first set of parameters is six.

In the embodiment shown in FIGS. 1-3, and within one “smooth pulse” or one pulse sequence Sₚ of the first laser system 2 operated with a first set of parameters, the fluences F of each individual pulse p of said one pulse sequence Sₚ accumulate to a cumulative fluence Fₛ. In such case the applied cumulative fluence Fₛ is preferably in a range from 2 J/cm², inclusive, to 150 J/cm², inclusive. In a preferred embodiment the applied cumulative fluence Fₛ is in a range from 3 J/cm², inclusive, to 25 J/cm², inclusive. In another preferred embodiment the applied cumulative fluence Fₛ is 9 J/cm².

In the embodiment shown in FIGS. 1-3 the sequence number M of subsequent “smooth pulse” pulse sequences Sₚ of the first laser system 2 operated with a first set of parameters is in a range from two, inclusive, to 20, inclusive. In a preferred embodiment the sequence number M of subsequent pulse sequences Sₚ of the first laser system 2 operated with a first set of parameters is in a range from two, inclusive, to four, inclusive. In another preferred embodiment the sequence number M of subsequent pulse sequences Sₚ of the first laser system 2 operated with a first set of parameters is three. In the embodiment shown in FIGS. 1-3 the sequence separation time T_{Ss} of the first laser system 2 operated with a first set of parameters is in a range from 0.2 s, inclusive, to 2 s, inclusive. In a preferred embodiment the sequence separation time T_{Ss} of the first laser system 2 operated with a first set of parameters is approximately 0.5 s.

In the embodiment shown in FIGS. 1-3 the sequence repetition time T_{Ss} of the first laser system 2 operated with a first set of parameters is in a range from 0.2 s, inclusive, to 2 s, inclusive. In a preferred embodiment the sequence repetition time T_{Ss} of the first laser system 2 operated with a first set of parameters is approximately 0.5 s.

The first laser system 2 of the laser device 1 referred to in FIGS. 1-3 can further be used to ablate the nail surface of the nail 8. This is in particular useful, when the nail 8 is significantly thicker than the average nail thickness s (FIG. 2), which is between 0.3 mm and 1 mm. The diffusion of heat from the surface of the nail 8 to the fungus layer 10 strongly depends on nail thickness. For thicker nails less heat is transmitted to the fungus layer 10. To have comparable conditions of treatments for each treatment, the non-ablating nail fungus laser treatment can be preceded by a nail ablating laser treatment. For the non-ablating nail fungus laser treatment the first laser system 2 is operated with the first set of parameters described above. For the preceding nail ablating laser treatment the first laser system 2 is operated with an ablating set of parameters.

When the strongly water-absorbed laser beam 3 of the first laser is applied to the infected nail 8 operated with the nail ablating set of parameters, these nail ablating parameters are adjusted such, that the infected nail 8 is irradiated in an ablating manner until the thickness s of the nail 8 is reduced to a value suitable for a subsequent non-ablating nail fungus laser treatment.

To ablate the nail surface of the nail 8 with the laser beam 3 of the first laser system 2 the crucial parameter is the fluence F of an individual pulse p. The fluence F must be above the ablation threshold of the nail 8. To achieve the desired ablation, the fluence F generated by one individual laser pulse p of the first laser system 2 operated with an ablating set of parameters within an irradiation spot 4 on the nail 8 is ≧2 J/cm². Preferably, said ablative fluence F is >10 J/cm².

As further schematically indicated in FIG. 2, and within the scope of the invention, the afore described non- or low ablative nail fungus treatment by means of the strongly water-absorbed laser beam 3 may be augmented by the application of the not strongly water-absorbed laser beam 7. The second laser system 6 of the laser device 1 referred to in FIGS. 1-2 is operated in a pulsed operation mode generating the non-water-absorbed laser beam 7 in individual pulses p with a second set of parameters. The laser parameters of said second parameter set are analogously defined as schematically shown in FIG. 3 with, however, absolute values and value ranges differing from the parameter values and value ranges as defined above for the water-absorbed laser beam 3.

In one preferred embodiment both laser beams 3, 7 are sequentially applied. In such case the same irradiation spot 4 on the nail 8 is first treated with at least a part of the pulse train of the water-absorbed laser beam 3. After an application separation time T_{AS} said water-absorbed application is then followed by the application of the non-water-absorbed laser beam 7 to the same irradiation spot 4 or at least an overlapped portion thereof. The application separation time T_{AS} is ≦1 s.

The advantage of this combined application of the laser beams of the first and the second laser system 2, 6 with laser beams 3, 7 with different wavelengths is that the temperature of the fungus layer 10 is first raised up by the water-absorbed laser beam 3 of the first laser system 2 to a temperature that is detrimental for the fungus without significantly affecting the temperature of the underlying nail bed 9, and thus creating a “temperature shock” to the fungus. Then the fungus is submitted to a second, “optical shock” by irradiating the fungus directly with the not strongly water absorbed laser beam 7 of the second laser system 6 that is nearly entirely transmitted through the nail 8 to the fungus layer 10, thus utilizing the additional germicidal effect of the direct illumination by the electromagnetic radiation. Since the fungus has been pre-heated, the optical shock treatment with the not strongly water-absorbed laser beam 7 of the second laser system 6 can be performed with parameter values causing a smaller degree of pain in the underlying nail bed 9 in comparison to the prior art, where a not strongly water-absorbed laser beam was applied directly to an infected nail without pre-heating of the nail by a strongly water-absorbed laser beam. Additionally, the treatment efficacy of the application of a not strongly water-absorbed laser beam according to prior art is enhanced due to the pre-heating.

In the alternative the first laser system 2 and the second laser system 6 of the laser device 1 referred to in FIGS. 1-3 can also be operated simultaneously. In this case the non- or low ablating nail fungus laser treatment by means of the water-absorbed laser beam 3 of the first laser system 2 is on the temporal scale at least partially overlapped by simultaneously applying the not strongly water-absorbed laser beam 7, generated by the second laser system 6, to the infected nail 8. As shown in FIG. 2 the irradiation spot 4 of the strongly water-absorbed laser beam 3 and the irradiation spot of the not strongly water-absorbed laser beam 7 are at least approximately identical. However, an at least partial spatial overlap of both on the nail 8 may suffice as well.

In both applications of the second laser system 6-simultaneous with the first laser system 2 or temporally separated from the first laser system 2-the second laser system 6 is operated in pulsed operation mode. The schematic intensity-time-profile shown in FIG. 3 also describes the intensity-time-profile of the laser beam 7 generated by the second laser system 6. In the following the same reference numbers and letters are used for the description of values and value ranges from which a second set of parameters is formed for the operation of the second laser system 6. In contrast to the application of the first laser system 2 the temporal separation between the individual pulses p is always constant. This means that the magnitudes of the pulse separation time tₚ, and of the sequence separation time T_{Ss} are equal for the application of the second laser system 6. Thus, the terms “multiple pulse sequences” and “sequence number M” are not applicable for describing the intensity-time-profile of the laser beam 7 of the second laser system 6. All individual pulses p of the second laser beam 7 build up one single pulse sequence Sₚ.

In the embodiment shown in FIGS. 1-3 the fluence F generated by one individual laser pulse p of the second laser system 6 operated with a second set of parameters within an irradiation spot 4 on the nail 8 is in a range from 1 J/cm², inclusive, to 150 J/cm², inclusive.

In the embodiment shown in FIGS. 1-3 the pulse duration tₚ of the second laser system 6 operated with a second set of parameters is in a range from 0.5 ns, inclusive, to 50 ms, inclusive.

### REFERENCES

Alley, M. R. K., Baker, S. J., Beutner, K. R., & Plattner, J. (2007). Recent progress on the topical therapy of onychomycosis. Expert Opinion on Investigational Drugs, 16(2), 157-167. doi:10.1517/13543784.16.2.157

Dias T. D., Steimacher A., Bento A. C., Neto A. M., Baesso M. L. (2007). Thermal characterization in vitro of human nail: photoacoustic study of the ageing process. Photochemistry and Photobiology, 83, 1144-1148.

Elewski, B. E. (1998). Onychomycosis: pathogenesis, diagnosis, and management. Clinical Microbiology Reviews, 11(3), 415-429.

Gupta, A., & Simpson, F. (2012). Device-based therapies for onychomycosis treatment. Skin Therapy Letter, 17(9), 4-9.

Ortiz, A. E., Avram, M. M., & Wanner, M. A. (214). A review of lasers and light for the treatment of onychomycosis. Lasers in Surgery and Medicine, 46(2), 117-124.

Tosti, A., Piraccini, B. M., Stinchi, C., & Colombo, M. D. (1998). Relapses of onychomycosis after successful treatment with systemic antifungals: a three-year follow-up. Dermatology (Basel, Switzerland), 197(2), 162-166.

Westerberg, D. P., & Voyack, M. J. (2013). Onychomycosis: Current trends in diagnosis and treatment. American Family Physician, 88(11), 762-770.

1. A method for treating a fungal infection in an infected nail, the method comprising:
placing an optical delivery system in the vicinity of the infected nail, wherein the optical delivery system is designed to deliver a laser beam from a laser system to the infected nail, and wherein the laser beam has a strongly water-absorbed wavelength in a range from above 1.9 μm to 11 μm, inclusive;
irradiating the diseased nail with the strongly water-absorbed laser beam, wherein a fluence and a duration of the laser irradiation received by the nail are adjusted such that by laser energy absorption in a surface portion of the nail a superficial heating of the nail and heat diffusion from said heated surface portion into the infected nail bed are achieved such that the bottom of the nail plate is heated to a treatment temperature needed to inactivate the infecting organism, wherein said treatment temperature is in a range from 40° C., inclusive, to 80° C., inclusive.

2. A method according to claim 1, wherein the nail is irradiated with the laser beam in the form of at least one individual laser pulse.

3. A method according to claim 2, wherein a pulse duration of the individual laser pulse is in range from 1 μs, inclusive, to 10 s, inclusive.

4. The method according to claim 2, wherein the fluence of the individual laser pulse is in a range from 0.2 J/cm², inclusive, to 150 J/cm², inclusive, preferably in a range from 0.5 J/cm², inclusive, to 10 J/cm², inclusive.

5. The method according to claim 2, wherein multiple individual pulses (p) are delivered in at least one pulse sequence (Sₚ), wherein one pulse sequence (Sₚ) has a pulse sequence duration (Tₛ), wherein within one pulse sequence (Sₚ) the individual pulses (p) are temporally separated by a pulse separation time (Iₚₛ) between the individual pulses (p), wherein the pulse sequence duration (Tₛ) is in range from 1 μs, inclusive, to 10 s, inclusive, and wherein the pulse separation time (tₚₛ) is ≧10 ms.

6. The method according to claim 5, wherein the cumulative fluence of the individual pulses within one pulse sequence is in a range from 2 J/cm², inclusive, to 150 J/cm², inclusive, preferably in a range from 3 J/cm², inclusive, to 25 J/cm², inclusive.

7. The method according to claim 5, wherein the pulse sequence duration (Tₛ) is in a range from 1 μs, inclusive, to 1.5 s, inclusive.

8. The method according to claim 5, wherein the pulse separation time (tₚₛ) is in a range from 0.01 s, inclusive, to 2 s, inclusive, and preferably in a range from 0.05 s, inclusive, to 0.2 s, inclusive.

9. The method according to claim 5, where the number of individual pulses within one pulse sequence is in a range from 4, inclusive, to 8, inclusive.

10. The method according to claim 5, wherein multiple pulse sequences follow one another with a sequence separation time, wherein the sequence separation time is in a range from 0.2 s, inclusive, to 2 s, inclusive

11. The method according to claim 10, wherein the number (M) of subsequent pulse sequences is in a range from 2, inclusive, to 20, inclusive, preferably in a range from 2, inclusive, to 4, inclusive.

12. The method according to claim 1, wherein the infected nail is heated to a treatment temperature in a range from 60° C., inclusive, to 80° C., inclusive.

13. The method according to claim 1, wherein said laser system is chosen from one of the following laser system types:
Er:YAG laser system generating a laser beam having a wavelength of 2.9 μm,
Tm:YAG laser system generating a laser beam having a wavelength of 2.0 μm,
Ho:YAG laser system generating a laser beam having a wavelength of 2.1 μm,
Erbium ion doped laser system, preferably Er,Cr:YSGG, Er:YSSG, Er:YAP or Er:YLF laser system, generating a laser beam having a wavelength in a range from 2.7 μm to 3.0 μm and
CO₂ laser system generating a laser beam having a wavelength in a range from 9.3 μm to 10.6 μm.

14. The method according to claim 1, wherein an irradiation spot (4) on the nail (8) is irradiated by the laser beam (3), and wherein a mean diameter of the irradiation spot is in a range of 4 mm, inclusive, to 8 mm.

15. The method according to claim 1, wherein a temperature sensing device, in particular an infrared temperature sensor, is used to monitor the temperature of the nail plate during treatment of the nail, and wherein the laser parameters are adjusted in response to a signal of the temperature sensing device to keep the nail temperature within a predefined treatment temperature range.

16. The method according to claim 1, wherein said method for treating a fungal infection in an infected nail is preceded by a nail ablating laser treatment using the strongly water-absorbed laser beam, wherein the laser beam is applied to the infected nail with laser parameters adjusted such that the infected nail is irradiated in an ablating manner until the thickness of the nail is reduced to a value suitable for a subsequent non-ablating nail fungus laser treatment.

17. The method according to claim 1, wherein said method for treating a fungal infection in an infected nail is followed by applying a not strongly water-absorbed laser beam, generated by a second laser system, to the infected nail, wherein the wavelength of said not strongly water-absorbed laser beam is in a range from 0.35 μm, inclusive, to 1.9 μm, inclusive, wherein the fluence of one individual laser pulse of said not strongly water-absorbed laser beam is in a range of 1 J/cm², inclusive, to 100 J/cm², inclusive, wherein the temporal pulse length of one individual laser pulse of said not strongly water-absorbed laser beam is in a range of 0.5 ns to 50 ms, and wherein an application separation time between the application of the water-absorbed laser beam and the non-water-absorbed laser beam is <1 s.

18. The method according to claim 1, wherein said method for treating a fungal infection in an infected nail is on the temporal scale at least partially overlapped by simultaneously applying a not strongly water-absorbed laser beam, generated by a second laser system, to the infected nail, wherein the wavelength of said not strongly water-absorbed laser beam is in a range from 0.35 μm, inclusive, to 1.9 μm, inclusive, wherein the fluence of one individual laser pulse of said not strongly water-absorbed laser beam is in a range of 1 J/cm², inclusive, to 100 J/cm², inclusive, wherein the temporal pulse length of one individual laser pulse of said not strongly water-absorbed laser beam is in a range of 0.5 ns to 50 ms.

19. A device for treating a nail fungal infection in a patient, the laser device comprising at least one laser system for generating a strongly water-absorbed laser beam having a wavelength in a range from above 1.9 μm to 11 μm, inclusive, and further comprising an optical delivery system, wherein the optical delivery system is designed to deliver the strongly water-absorbed laser beam from the laser system to the infected nail, and wherein the device is adapted to provide a fluence and a duration of the laser irradiation received by the nail being adjusted such, that by laser energy absorption in a surface portion of the nail a superficial heating of the nail and heat diffusion from said heated surface portion into the infected nail bed are achieved such, that the bottom of the nail plate is heated to a treatment temperature needed to inactivate the infecting organism, wherein said treatment temperature is in a range from 40° C., inclusive, to 80° C., inclusive.

20. A method of making a device for treating a nail fungal infection in a patient, the laser device comprising at least one laser system for generating a strongly water-absorbed laser beam having a wavelength in a range from above 1.9 μm to 11 μm, inclusive, and further comprising an optical delivery system, wherein the optical delivery system is designed to deliver the strongly water-absorbed laser beam from the laser system to the infected nail, and wherein the device is adapted to provide a fluence and a duration of the laser irradiation received by the nail being adjusted such, that by laser energy absorption in a surface portion of the nail a superficial heating of the nail and heat diffusion from said heated surface portion into the infected nail bed are achieved such, that the bottom of the nail plate is heated to a treatment temperature needed to inactivate the infecting organism, wherein said treatment temperature is in a range from 40° C., inclusive, to 80° C., inclusive.
